(19) United States
(12) Patent Application Publication
(10) Pub. No.: US 2004/0023937 A1
(43) Pub. Date: Feb. 5, 2004

(54) Anti-cancer formulation

(75) Inventor: Stephen H. Monroe, Memphis, TN (US)

Correspondence Address: PITTS AND BRITTIAN P C, P O BOX 51295, KNOXVILLE, TN, 37950-1295, US

(73) Assignee: Greystone Medical Group, Inc., Memphis, TN (US)

(21) Appl. No.: 10/443,476

(22) Filed: May 22, 2003

Related U.S. Application Data
(60) Provisional application No. 60/382,460, filed on May 24, 2002.

(51) Int. Cl. A61K 35/78; A61K 31/573

(52) U.S. Cl. 514/179; 424/779

(57) ABSTRACT

Composition for halting the metastasis of cancer cells comprising rubidum ursolate. The rubidium ursolate is formed from a mixture of rubidium and ursolic acid obtained from the bark of the Virburnum plant.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of Invention

This invention relates to formulations for the preventing or halting the metastasis of cancer cells.

### DETAILED DESCRIPTION OF THE INVENTION

Ursolic acid derived from the bark of the Viburnum plant has been reported to have anti-invasive activity with human fibrosarcoma cells. The mechanism of activity was reported to be thought to involve the down-regulation of MMP-9, a gelatin degrading matrix metalloproteinase. It has been further reported that there was no effect on MMP-2, the second of two gelatin degrading MMP's.

In accordance with the present invention, Rubidium is combined with Ursolic acid to make a salt of the acid, Rubidium Ursolate, which, when administered to a patient, down-regulates both MMP-2 and MMP-9. This combination of the two MMP inhibitors provides a treatment for halting the spread or metastasis of cancer cells, in general.

Having thus described the aforementioned invention, we claim:

1. A composition for halting the metastasis of cancer cells comprising pharmaceutically effective amounts of rubidium ursolate.

2. The composition of claim 1 wherein said composition, when administered to a patient, down-regulates both MMP-2 and MMP-9.

3. The composition of claim 1 wherein said rubidium ursolate is formed from a mixture of rubidium and ursolic acid.

4. The composition of claim 3 wherein said ursolic acid is obtained from the bark of the Virburnum plant.

5. A method for halting the metastasis of cancer cells comprising administration to a patient in need thereof of a pharmaceutically effective amount of rubidium ursolate.

6. The method of claim 5 whereins said rubidium ursolate is formed from a mixture of ursolic acid and rubidium.

7. The method of claim 5 wherein said ursolic acid is derived from the bark of the Virburnum plant.
